(19) United States
(12) Patent Application Publication
(10) Pub. No.: US 2013/0150877 A1
(43) Pub. Date: Jun. 13, 2013

(54) TREATMENT INSTRUMENT

(71) Applicant: OLYMPUS CORPORATION, Tokyo (JP)

(72) Inventor: Hiromu IKEDA, Tokyo (JP)

(73) Assignee: OLYMPUS CORPORATION, Tokyo (JP)

(21) Appl. No.: 13/760,153

(22) Filed: Feb. 6, 2013

Related U.S. Application Data
(63) Continuation of application No. PCT/JP2011/068260, filed on Aug. 10, 2011.

(30) Foreign Application Priority Data
Aug. 11, 2010 (JP) 2010-180298

(51) Int. Cl. A61B 17/34

(52) U.S. Cl. CPC A61B 17/34
USPC 606/185

(57) ABSTRACT

Provided is a treatment instrument including a guide member with a tip surface to be pressed against an outer surface of a pericardium; a pericardium fixing member including at least one needle-shaped portion having a sharp pointed end extending diagonally forward along a circumferential direction at a tip of a cylinder-shaped body rotatable around the axis thereof; a perforating member for forming a through-hole for a guide wire in the pericardium to which the pericardium fixing member is fixed; and a pointed end position restricting portion for restricting the position of the pointed end in a direction of the axis relative to the tip surface of the guide member at a location where the pointed end of the pericardium fixing member does not reach the outer surface of a heart while the tip surface of the guide member is pressed against the outer surface of the pericardium.

### TECHNICAL FIELD

The present invention relates to a treatment instrument.

### BACKGROUND ART

Conventionally, there have been known treatment instruments which perforate a pericardium and insert a guide wire therethrough (For example, see Patent Literatures 1 and 2).

The treatment instrument of Patent Literature 1 includes a needle-shaped protrusion extending in a circumferential direction at a tip of a cylindrical body. With the needle-shaped protrusion butted on the surface of a pericardium, cylindrical body is rotated around the axis of the cylindrical body. In such a way, the needle-shaped protrusion is stuck into the pericardium to fix the cylindrical body in the pericardium, and the cylindrical body is raised to form a space between the pericardium and a heart.

At a location in a space formed in this way, a pericardium is perforated by an access device for perforation to prevent a heart from being damaged when perforated.

Moreover, a treatment instrument of Patent Literature 2 also attempts to prevent damage to the heart by slicking a cylindrical body with a tip opening pressed against a pericardium to raise the pericardium, and then by perforating the pericardium.

### CITATION LIST

### Patent Literature

{PTL 1} U.S. Pat. No. 6,423,051
{PTL 2} Japanese Translation of PCT International Application, Publication No. 2001-516625

### SUMMARY OF INVENTION

### Technical Problem

In the treatment instrument of Patent Literature 1, a space formed in a pericardium may be smaller than an insertion amount of an access device for perforation. In such a case, the tip of the access device may cause damage to myocardium and a blood vessel of the heart.

Moreover, in the treatment instrument in Patent Literature 2, the variation in the pressure inside a cylindrical body for forming a space causes the size of the space to be changed. For example, the imperfect sealing condition between the cylindrical body and the pericardium causes surrounding air to enter the cylindrical body, or air enters the cylindrical body through a hole in the pericardium formed by the access device. This decreases a decompression condition in the cylindrical body to reduce a space formed in the pericardium. Even in this case, the access device may cause damage to myocardium and the like of the heart.

The present invention has been made in view of the above-described circumstance, and it is an object thereof to provide a treatment instrument capable of surely preventing damage to a heart within a pericardium in perforation of the pericardium and insertion of a guide wire.

### Solution to Problem

The present invention provides a treatment instrument, including: a guide member with a tip surface to be pressed against an outer surface of a pericardium; a pericardium fixing member including at least one needle-shaped portion having a sharp pointed end extending diagonally forward along a circumferential direction at a tip of a cylinder-shaped body rotatable around the axis of the cylinder-shaped body, wherein the cylinder-shaped body is rotated around the axis so as to stick the pointed end of the needle-shaped portion into the pericardium to fix the cylinder-shaped body to the pericardium; a perforating member for forming a through-hole for a guide wire in the pericardium to which the pericardium fixing member is fixed; and a pointed end position restricting portion for restricting the position of the pointed end in a direction of the axis relative to the tip surface of the guide member at a location where the pointed end of the pericardium fixing member does not reach the outer surface of a heart while the tip surface of the guide member is pressed against the outer surface of the pericardium (first aspect).

In the first aspect, the treatment instrument may include: a perforating member position restricting portion for restricting the position of the perforating member in the direction of the axis relative to the tip surface of the guide member at a location where the perforating member does not reach the outer surface of the heart while the tip surface of the guide member is pressed against the outer surface of the pericardium (second aspect).

In the first aspect, the guide member may be formed into a cylindrical shape into which the pericardium fixing member is inserted (third aspect).

In the first aspect, the needle-shaped portion may be formed into a spiral shape (fourth aspect).

In the first aspect, the pericardium fixing member may be provided so as to be movable relatively in the direction of the axis with respect to the guide member, and the pointed end position restricting portion may comprise an abutment surface provided on the pericardium fixing member to butt the guide member (fifth aspect).

In the second aspect, the pericardium fixing member may be provided so as to be movable relatively in the direction of the axis with respect to the guide member; the perforating member may be provided so as to movable relatively in the direction of the axis with respect to the guide member or the pericardium fixing member; the pointed end position restricting portion may include an abutment surface provided on the pericardium fixing member to butt the guide member; and the perforating member position restricting portion may include another abutment surface provided on the perforating member to butt the guide member or the pericardium fixing member (sixth aspect).

In the fourth aspect, the perforating member may be fixed to the pericardium fixing member, with a sharp tip of the perforating member arranged posterior to the pointed end of the needle-shaped portion (seventh aspect).

In the seventh aspect, the perforating member may be a linear needle-shaped member arranged at the center of the spiral needle-shaped portion along the axis (eighth aspect).

In the seventh aspect, the perforating member may be a spiral needle-shaped member arranged coaxially with the needle-shaped portion having the spiral shape (ninth aspect).

In the first aspect, the guide member, the pericardium fixing member, and the perforating member may be each provided with a bendable portion allowing tip portions of the members to be bent simultaneously in the same direction (tenth aspect).

In the seventh aspect, the guide member and the pericardium fixing member may be each provided with a bendable portion allowing tip portions of the members to be bent simultaneously in the same direction (eleventh aspect).

### BRIEF DESCRIPTION OF DRAWINGS

FIG. 1 is a whole configuration view showing a state in which the tip of a treatment instrument according to one embodiment of the present invention is placed in contact with a pericardium via a trocar.

FIG. 2 is a longitudinal sectional view showing the tip portion of the treatment instrument in FIG. 1.

FIG. 3 is a view of a pericardium fixing member of the treatment instrument in FIG. 1 when viewed from the tip portion side in a direction along the axis thereof.

FIG. 4 is an enlarged view showing a needle-shaped portion provided at the tip of the pericardium fixing member of the treatment instrument in FIG. 1.

FIG. 5 is a view showing a state with an outer sheath of the treatment instrument in FIG. 1 inserted into the trocar.

FIG. 6 is a longitudinal sectional view showing a state with a tip surface of the outer sheath in the state in FIG. 5 pressed against the surface of the pericardium.

FIG. 7 is a view showing a state with the pericardium fixing member inserted in the outer sheath in FIG. 5.

FIG. 8 is a longitudinal sectional view of the tip portion showing a state with the pericardium fixing member inserted in the outer sheath in FIG. 6 to a maximum extent.

FIG. 9 is a longitudinal sectional view of the tip portion showing a state with the needle-shaped portion passed through the pericardium after rotation of the pericardium fixing member around the axis from the state in FIG. 8.

FIG. 10 is a view showing a state with a perforating member inserted in the pericardium fixing member in FIG. 8.

FIG. 11 is a longitudinal sectional view showing the tip portion of a treatment instrument in FIG. 10.

FIG. 12 is a longitudinal sectional view of the tip portion showing a state with the perforating member inserted in the pericardium fixing member of the treatment instrument in FIG. 11 to a maximum extent, and the perforating member passed through the pericardium.

FIG. 13 is a longitudinal sectional view of the tip portion showing a state with the tip of a guide wire inserted in a cardiac sac via the perforating member in the state in FIG. 12.

FIG. 14 is a longitudinal sectional view of the tip portion showing a state with the perforating member removed from the inside of the pericardium fixing member in the state in FIG. 13.

FIG. 15 is a longitudinal sectional view of the tip portion showing a state with the pericardium fixing member removed from the inside of the outer sheath in the state in FIG. 14.

FIG. 16 is a view showing a state with the outer sheath in FIG. 14 removed and only the guide wire detained via the trocar.

FIG. 17 is a longitudinal sectional view showing a modification of the treatment instrument in FIG. 1.

FIG. 18 is a longitudinal sectional view of a tip portion showing other modification of the treatment instrument in FIG. 1.

FIG. 19 is a longitudinal sectional view of a tip portion showing other modification of the treatment instrument in FIG. 1.

FIG. 20 is a longitudinal sectional view of a tip portion showing other modification of the treatment instrument in FIG. 1.

FIG. 21 is a longitudinal sectional view of a tip portion showing a state with a bendable portion bent, the bendable portion being provided in the tip of the treatment instrument in FIG. 20.

FIG. 22 is a view showing a state with the tip of the treatment instrument in FIG. 20 brought into intimate contact with the pericardium.

### DESCRIPTION OF EMBODIMENTS

A treatment instrument according to one embodiment of the present invention will be described below with reference to drawings.

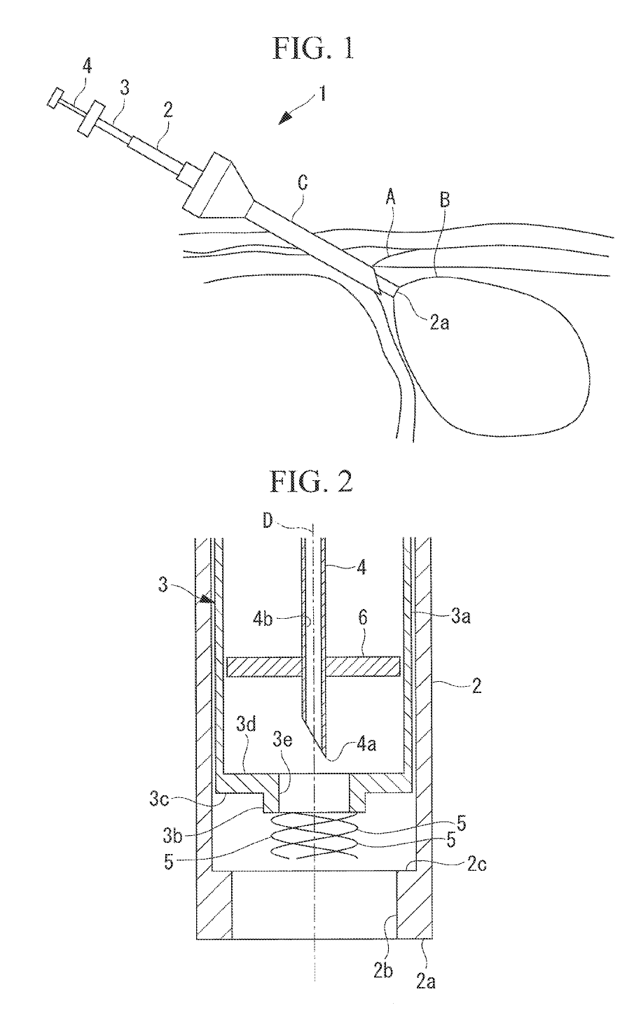

A treatment instrument 1 according to the present embodiment, as shown in FIG. 1, is an instrument inserted into a body via a trocar C, a tip of which is arranged so as to face an outer surface of a pericardium B through the abdomen under a saber-shaped protrusion A. The treatment instrument 1 includes an elongated cylinder-shaped outer sheath (guide member) 2, a pericardium fixing member 3 inserted into the outer sheath 2, and a perforating member 4 further inserted thereinto.

The outer sheath 2, as shown in FIG. 1, has a slightly smaller outer diameter dimension than the inner diameter dimension of the trocar C. By inserting the outer sheath 2 via the trocar C, the outer sheath 2 may press a tip surface 2a against the outer surface of the pericardium B. As shown in FIG. 2, a small diameter portion 2b having an inner diameter dimension formed into a still smaller diameter is provided on the tip side of the outer sheath 2. The small diameter portion 2b has a step portion 2c protruding in a radially inner direction within the outer sheath 2.

A pericardium fixing member 3 is an elongated member which may be inserted into the inside of the outer sheath 2, the pericardium fixing member 3 including a body having a cylindrical shape (a cylinder-shaped body) 3a, and needle-shaped portions 5 mounted on a tip of the body 3a. The body 3a is somewhat smaller than the inner diameter dimension of the outer sheath 2, and has an outer diameter dimension that is larger than the small diameter portion 2b of the outer sheath 2. The tip of the body 3a is provided with a projection portion 3b with the outer diameter dimension formed into still smaller dimension.

A shoulder is formed between the projection portion 3b and the body 3a. The shoulder constitutes an abutment surface 3c which butts against the step portion 2c provided in the outer sheath 2. Thus, when the pericardium fixing member 3 is inserted into the outer sheath 2, the abutment surface 3c of the pericardium fixing member 3 butts against the step portion 2c inside the outer sheath 2, thereby restricting further insertion of the pericardium fixing member 3. Moreover, the shoulder forming the external abutment surface 3c constitutes a step portion 3d projecting from the body 3a in a radially inner direction within the body 3a of the pericardium fixing member 3. Further, a through-hole 3e is provided in the center of the projection portion 3b.

In this way, the outer sheath is arranged at a location where the outer periphery of the pericardium fixing member is surrounded, and therefore, as described below, it is possible to stabilize the operation of the pericardium fixing member and support it in fixing working to the pericardium using the pericardium fixing member.

Needle-shaped portions 5 are fixed to the tip of the projection portion 3b of the pericardium fixing member 3.

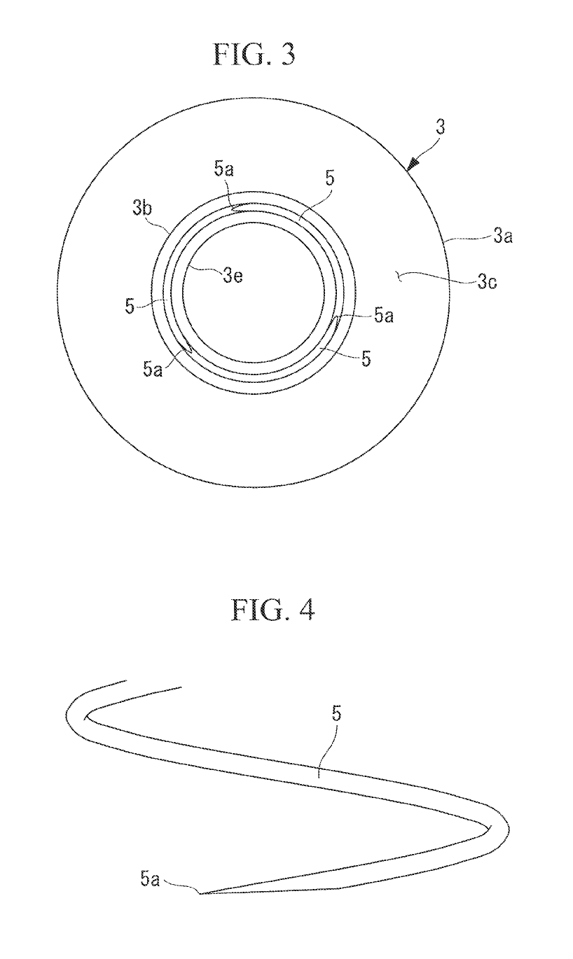

The needle-shaped portions 5, as shown in FIG. 3, are spiral members arranged around an axis D of the body 3a. Three needle-shaped portions 5 are spaced in a circumferential direction. A sharp pointed end 5a of each needle-shaped portion 5 is arranged in a circumferential direction, and as shown in FIG. 4, is arranged diagonally forward. The needle-shaped portions 5, for example, as shown in FIG. 3, are each arranged coaxially with the same radial dimension, and formed into a spiral having the same lead in the same direction.

In such a configuration, one or more sharp pointed ends of the needle-shaped portions provided at the tip of the cylinder-shaped body constituting the pericardium fixing member are brought into contact with the surface of the pericardium, and the cylinder-shaped body is rotated around its axis. Thus, the pointed ends of the needle-shaped portions extending diagonally forward along a circumferential direction stick in the pericardium. By further rotating the cylinder-shaped body around the axis, the needle-shaped portions may be deeply stuck to fix the cylinder-shaped body to the pericardium.

The sharp pointed ends 5a of the needle-shaped portions 5, as shown in FIG. 8, are positioned at a somewhat retracted location toward the rear from the tip surface 2a of the outer sheath 2 in a direction of the axis D with the abutment surface 3c butted on the step portion 2c of the outer sheath 2. Thus, as shown in FIG. 8, when the abutment surface 3c of the pericardium fixing member 3 is inserted until it butts against the step portion 2c of the outer sheath 2 while the tip surface 2a of the outer sheath 2 is pressed against the outer surface of the pericardium B, the pointed end 5a of the needle-shaped portion 5 is adapted to be positioned at a location where the pointed end 5a is in contact with the outer surface of the pericardium B, but does not pass through the pericardium B.

At this time, the tip surface of the outer sheath is pressed against the outer surface of the pericardium, and the axial direction position of the pointed ends of the needle-shaped portions relative to the tip surface of the outer sheath is restricted in a location where they do not reach the outer surface of the heart by the step portion. Therefore, even when the cylinder-shaped body is rotated to proceed with centesis into the pericardium by the needle-shaped portions, the pointed ends are maintained in any position without contact with the outer surface of the heart. Consequently, the cylinder-shaped body may be fixed to the pericardium without causing damage to the heart.

It is noted that the position of the pointed end 5a is defined by the curvature of the pericardium B at a site where the tip surface 2a of the outer sheath 2 of the treatment instrument 1 is butted. For example, when a site where the tip surface 2a is butted is in a convex surface having a large curvature, the sharp pointed ends 5a of the needle-shaped portions 5 are positioned at a retracted location more rearward in a direction of the axis D than in a convex surface having a small curvature. When a site where the tip surface 2a is butted is in a substantially flat surface, the pointed ends 5a may be positioned at the substantially same location in direction of the axis D as the tip surface 2a of the outer sheath 2.

The perforating member 4 is formed into a cylindrical shape which may be inserted inside the pericardium fixing member 3. A tip 4a is formed into an injection needle shape that is obliquely cut. The perforating member 4 has a somewhat larger inner diameter dimension than the outer diameter dimension of a guide wire E such that the guide wire E (see FIG. 13) may be inserted. The perforating member 4 is adapted to be arranged linearly along the axis in the center in a radial direction of the pericardium fixing member 3.

In this manner, a space is formed between a pericardium and a heart while the cylinder-shaped body is fixed to the pericardium, and the perforating member forms a through-hole for a guide wire in the pericardium, and therefore the guide wire may be easily inserted from outside the pericardium to inside the pericardium via the through-hole.

A disk-shaped jaw member 6 (another abutment surface) extending in a radially outer direction is fixed to the perforating member 4. The outer diameter dimension of the jaw member 6 is formed in smaller size than the inner diameter dimension of the body 3a of the pericardium fixing member 3, and in larger size than the inner diameter dimension of the projection portion 3c. In this configuration, when the perforating member 4 is inserted into the pericardium fixing member 3, the jaw member 6 butts against the step portion 3d in the pericardium fixing member 3 so as to restrict further insertion of the perforating member 4.

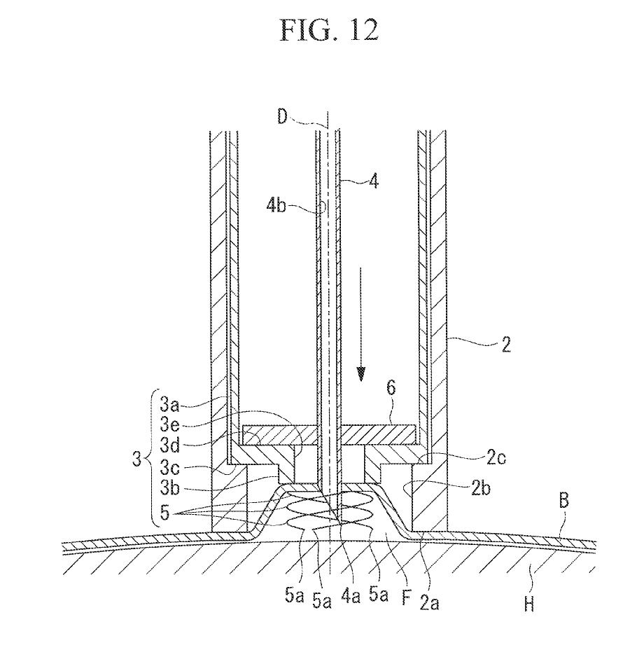

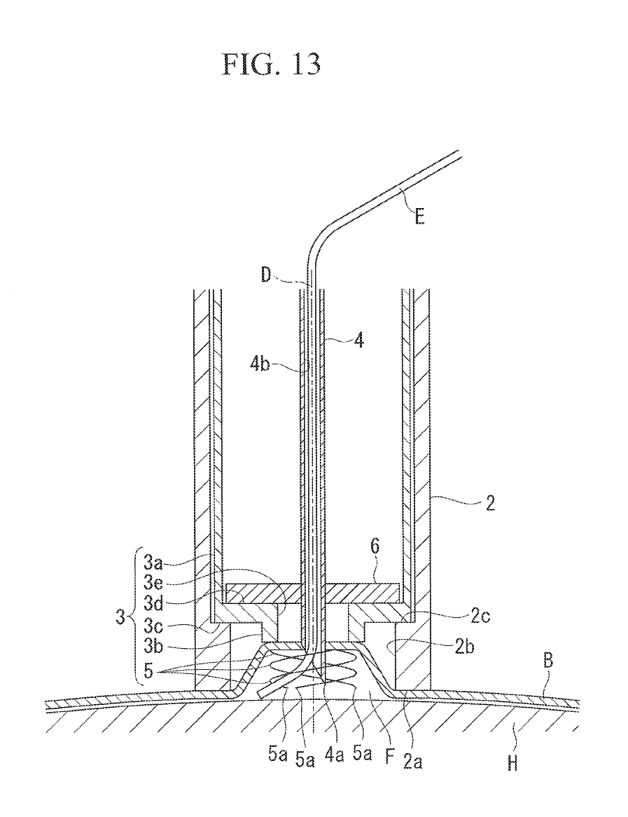

As shown in FIG. 12, the tip 4a of the perforating member 4 is adapted to be positioned at a (retracted) location more rearward in a direction of the axis D than the pointed end 5a of the needle-shaped portion 5 with the jaw member 6 butted against the step portion 3d of the pericardium fixing member 3.

In this way, due to the step portion, the position of an axial direction of the perforating member relative to the tip surface of the outer sheath pressed against the outer surface of the pericardium is restricted in a location where it does not reach the outer surface of the heart. Therefore, even when the perforating member forms a through-hole into the pericardium, the perforating member is not brought into contact with the heart so that the perforating member may easily perforate a through-hole for a guide wire without causing damage to the heart.

The working of a treatment instrument 1 according to the present embodiment as constituted in this way will be described below.

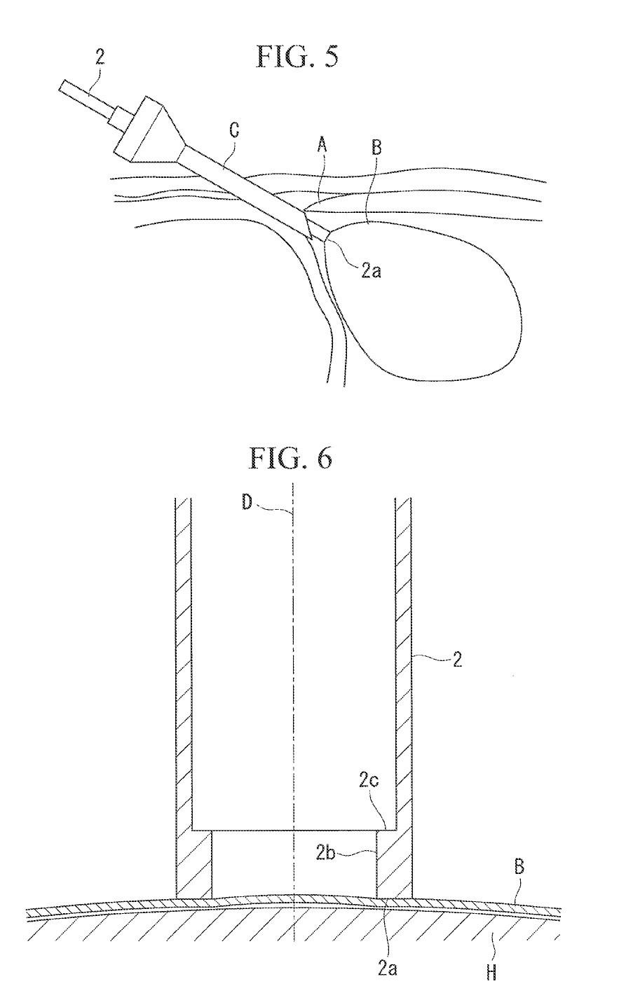

In order to insert a guide wire E into a space (a cardiac sac) F between a pericardium B and a heart H using the treatment instrument 1 according to the present embodiment, as shown in FIGS. 5 and 6, the outer sheath 2 is inserted into the trocar C inserted in position through the abdomen under a saber-shaped protrusion A, the tip surface 2a of the outer sheath 2 being pressed against the outer surface of the pericardium B.

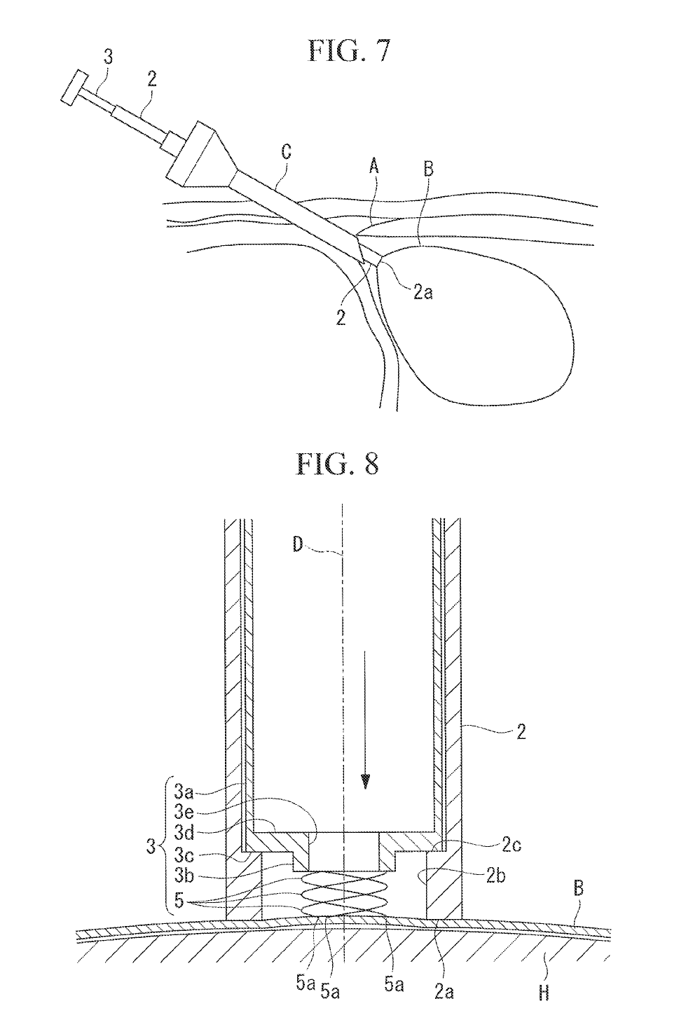

In this condition, as shown in FIG. 7, when the pericardium fixing member 3 is inserted into the outer sheath 2, as shown in FIG. 8, the abutment surface 3c provided in the vicinity of the tip of the pericardium fixing member 3 butts against the step portion 2c in the outer sheath 2, thereby restricting further insertion. At this time, the sharp pointed ends 5a of the needle-shaped portions 5 provided at the tip of the pericardium fixing member 3 are in contact with the surface of the pericardium B, but positioned at a location where they do not pass through the pericardium B.

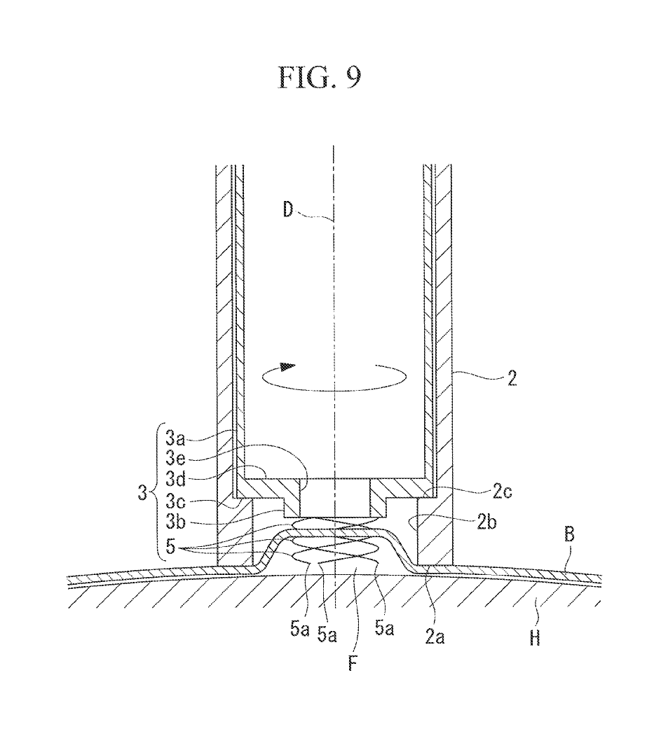

Next, as shown in FIG. 9, the pericardium fixing member 3 is rotated in one direction around its axis D, while the outer sheath 2 is fixed. This causes the pointed ends 5a of the needle-shaped portions 5, which is in contact with the surface of the pericardium B, to be moved in a circumferential direction around the axis D to conduct centesis into the pericardium B.

Since the needle-shaped portions 5 are formed into the spiral, the pointed ends 5a of the needle-shaped portions 5 conduct deep centesis into the pericardium B with rotation of the pericardium fixing member 3. With further rotation, the pointed ends 5a passes through the pericardium B. When the needle-shaped portions 5 are rotated, the pericardium B is moved in a direction away from the surface of the heart H by the spiral needle-shaped portions 5 to expand a space F between the pericardium B and the heart H, as shown in FIG. 9. This allows the pericardium fixing member 3 to be fixed to the pericardium B.

In other words, the cylinder-shaped body of the pericardium fixing member is rotated to stick the pointed ends of the needle-shaped portions into the pericardium, and in such a state the cylinder-shaped body is rotated. This allows the needle-shaped portions formed in the spiral to further deeply stick into the pericardium, so that the pericardium is pulled up in the axial direction of the cylinder-shaped body, in a direction apart from the heart, according to the spiral lead. Therefore, the space between the heart and the pericardium may be formed just by rotating the cylinder-shaped body, allowing for easy perforation working by the perforating member.

In such a case, for the pointed end 5a of the needle-shaped portion 5, the location in the direction of the axis D thereof is restricted based on butting of the abutment surface 3c of the pericardium fixing member 3 against the step portion 2c in the outer sheath 2. Therefore, even if the pointed end 5a of the needle-shaped portion 5 passes through the pericardium B, they are maintained in any position without contact with the surface of the heart H. As a result, this prevents the pointed end 5a of the needle-shaped portion 5 from causing damage to the surface of the heart H.

Due to this, when the pericardium fixing member is moved in the axial direction relative to the outer sheath having its tip surface pressed against the outer surface of the pericardium, the abutment surface provided in the pericardium fixing member butts against the outer sheath, so that the pointed ends of the needle-shaped portions is restricted in an axial direction position where they do not reach the surface of the heart. This allows for simple fixing working to the pericardium by the pericardium fixing member.

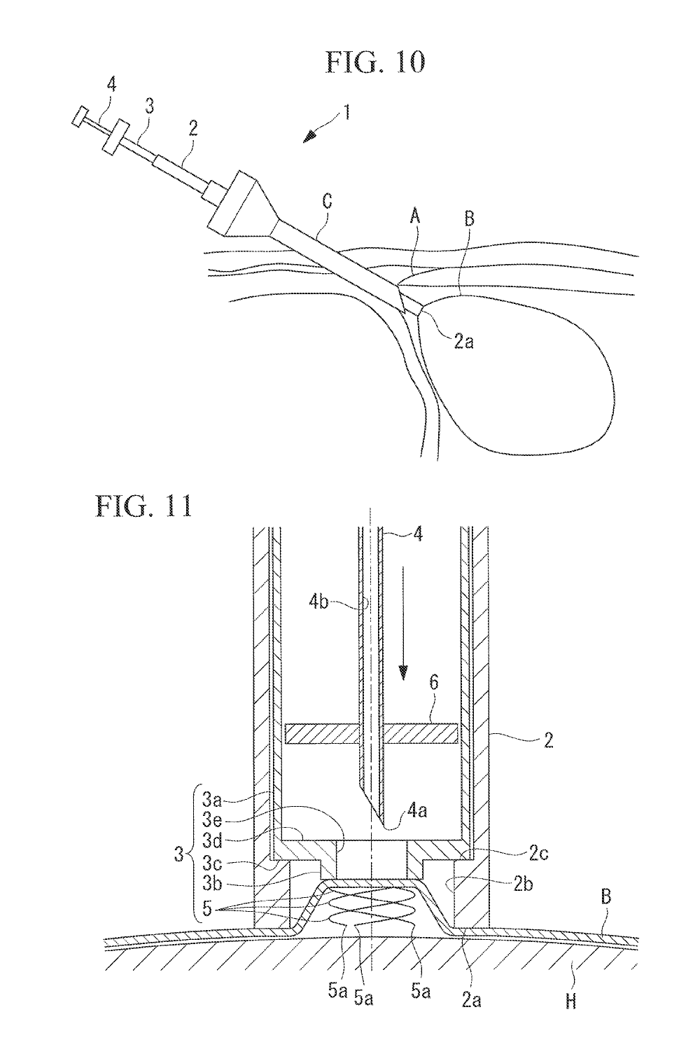

Then, as shown in FIGS. 10 and 11, the perforating member 4 is inserted into the body 3a of the pericardium fixing member 3, and the sharp tip 4a is brought close to the pericardium B fixed to the tip of the pericardium fixing member 3, and as shown in FIG. 12, the sharp tip 4a is passed through the pericardium B. Since the pericardium B is away from the surface of the heart H using the spiral needle-shaped portions 5, the tip 4a of the perforating member 4 which has passed through the pericardium B is positioned within this space F.

In particular, the jaw member 6 is provided to the perforating member 4. Therefore, by butting the jaw member 6 against the step portion 3d of the pericardium fixing member 3, the location in a direction of the axis D of the sharp tip 4a is restricted in a retracted location more rearward in a direction of the axis D than the pointed end 5a of the needle-shaped portion 5. As described above, the needle-shaped portion 5 is restricted in a location without contact with the heart H, and therefore the tip 4a of the perforating member 4 is also restricted in a location without contact with the heart H, so that the perforating member 4 passing through the pericardium B to form the through-hole does not cause damage to the heart H.

In other words, when the perforating member is moved in its axial direction relative to the outer sheath having the tip surface pressed against the outer surface of the pericardium, or relative to the pericardium fixing member with the abutment surface butted against the outer sheath, another abutment surface provided in the perforating member butts against the outer sheath or the pericardium fixing member, so that the perforating member is restricted in an axial direction position at which the perforating member does not reach the surface of the heart. This allows for easy perforation working of the through-hole into the pericardium by the perforating member.

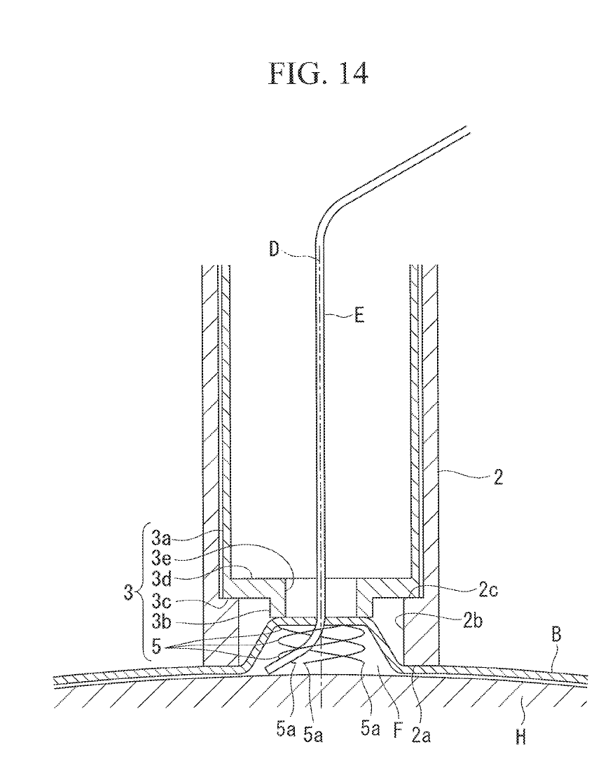

The perforating member 4 is formed into a hollow shape such as an injection needle, and therefore, as shown in FIG. 13, the guide wire E may be inserted into the cardiac sac F through a central hole 4b of the perforating member 4 with a through-hole formed through the pericardium B. Then, as shown in FIG. 14, the perforating member 4 is retracted relative to the pericardium fixing member 3 to be capable of pulling up the perforating member 4.

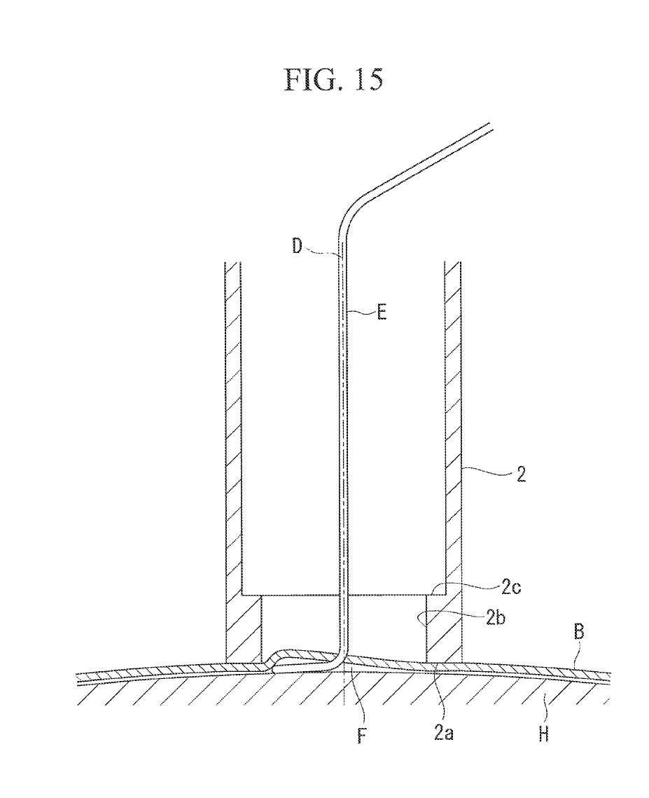

Further, by rotating the pericardium fixing member 3 in the opposite direction as the above relative to the outer sheath 2, the needle-shaped portions 5 which has been passed through the pericardium B are removed from the pericardium B, the needle-shaped portions 5 being capable of being removed from the inside of the outer sheath 2, as shown in FIG. 15.

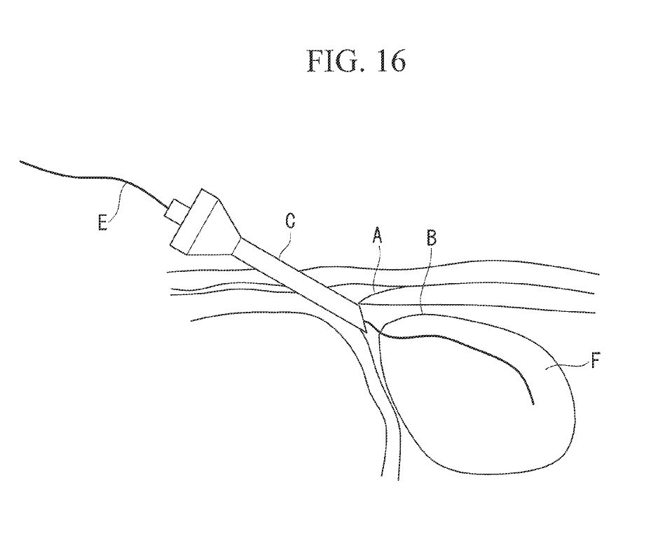

As shown in FIGS. 15 and 16, this allows only the guide wire E to be left in a state inserted in the cardiac sac F through the through-hole formed in the pericardium B.

Thus, the treatment instrument 1 according to the present embodiment has an advantage of capable of more surely preventing the heart H within the pericardium B from being subjected to damage in perforate of the pericardium B and in insertion of the guide wire E, and of easily inserting the guide wire E into the cardiac sac F with easy operation.

It is noted that in the present embodiment, three spiral needle-shaped portions 5 are concentrically arranged, and however, instead of this, any one or more needle-shaped portions 5 may be adopted. As the number thereof increases, a strain that is imposed on the pericardium B is reduced. However, about three needle-shaped portions are desired since the number of the holes formed into the pericardium B increases.

In many cases, the thickness of the pericardium usually is about 0.5 to 2 mm. However, in the case where, e.g., once the pericardium is lost due to such as any disease or surgery, the thickness of the pericardium may be about 0.05 mm, which is very thin, or may be about 10 mm due to such as accumulation of fat and pericarditis.

The step portion 2c and the abutment surface 3c may restrict the amount of protruding of the pericardium fixing member 3 from the outer sheath 2 not more than the thickness of the pericardium B. However, since there are great differences between individuals for the thickness of the pericardium B as seen above, it is desirable to previously measure the thickness of the pericardium B in an insertion site before insertion of the pericardium fixing member 3.

A method of previously measuring the thickness of the pericardium B with CT scan, MRI, or the like, or a method of measuring it with ultrasound echo measurements, optical coherence tomography, and the like after access to the pericardium is suitable for measuring the thickness of the pericardium B.

Moreover, since a site of the heart H that contacts the outer sheath 2 has a convex shape, the amount of protruding of the pericardium fixing member 3 defined by the step portion 2c and the abutment surface 3c may be within the tip of the outer sheath 2, and the amount of protruding thereof is comprehensively determined based on the shape and size of the heart H, and the thickness of the pericardium B.

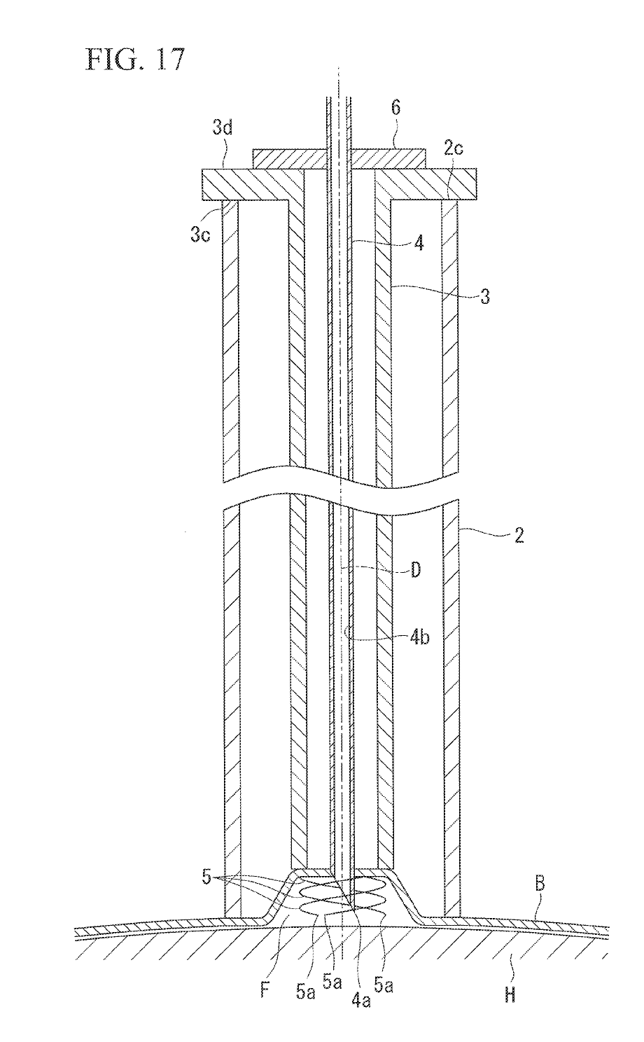

In the present embodiment, the step portions 2c, 3d, the abutment surface 3c, and the jaw member 6 are provided on the outer sheath 2, the pericardium fixing member 3, and the pointed end side of the perforating member 4, respectively. Thus, they are butted such that the pointed end 5a of the needle-shaped portion 5 and the tip 4a of the perforating member 4 are in no contact with the heart H, but instead of this, as shown in FIG. 17, the outer sheath 2 and the pericardium fixing member 3, and the pericardium fixing member 3 and the perforating member 4 may be butted on their base end side.

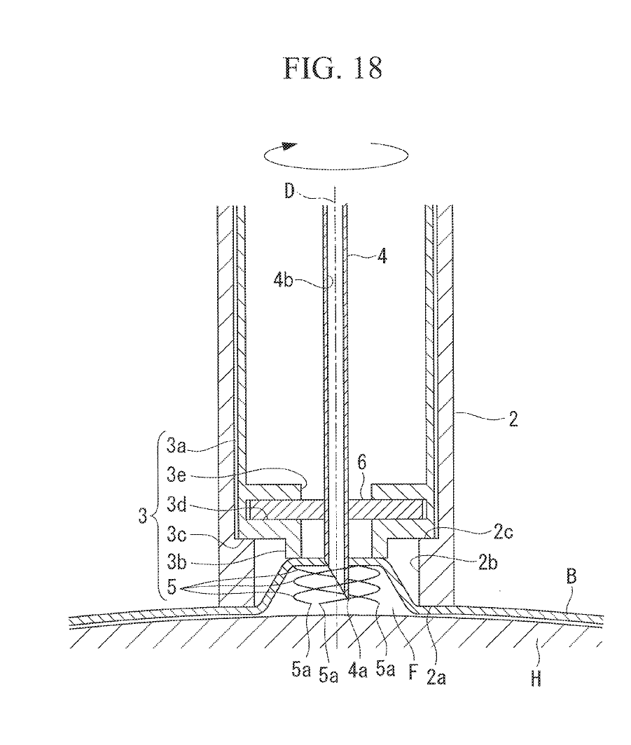

Moreover, in the present embodiment, the perforating member 4 is provided relatively movably in a direction of the axis D relative to the pericardium fixing member 3, but instead of this, as shown in FIG. 18, the perforating member 4 may be fixed in a direction of the axis D relative to the pericardium fixing member 3. In the above embodiment, the positional relationship between the pointed end 5a of the needle-shaped portion 5 and the tip 4a of the perforating member 4 may be the same as that in the case where the jaw member 6 are butted against the step portion 3d. The perforating member 4 preferably is mounted rotatably around the axis D relative to the pericardium fixing member 3.

In this way, when the pericardium fixing member 3 is rotated around the axis to move the pericardium B away from the heart H using the spiral needle-shaped portions 5, the pericardium B gets close to the body 3a of the pericardium fixing member 3, therefore such that the perforating member 4 fixed to the body 3a passes through it. In this case, the perforating member 4 is maintained so as not to rotate, regardless of the rotation of the pericardium fixing member 3. Therefore, the perforating member 4 is quietly stuck into the pericardium B to be capable of easily form a through-hole without largely causing damage to the pericardium B.

In this way, the cylinder-shaped body is rotated to rotate the spiral needle-shaped portions around the axis, thereby sticking the pointed ends of the needle-shaped portions to the pericardium, and moving the pericardium in a direction away from the surface of the heart according to the spiral of the needle-shaped portion. By doing so, the pericardium is brought close to the sharp tip of the perforating member fixed to the pericardium fixing member, and the sharp tip sticks in the pericardium, so that a through-hole is formed in the pericardium.

In this case, the tip of the perforating member is positioned backward of the pointed ends of the needle-shaped portions in the axial direction. Therefore, this may surely prevent the perforating member from being brought into contact with the heart and causing damage to the heart. Moreover, the tip surface of the guide member is pressed against the outer surface of the pericardium, and the abutment surface of the pericardium fixing member is butted against the guide member. In such a state, just by rotating the pericardium fixing member around the axis, a through-hole may easily be perforated in the pericardium using the perforating member.

Further, even when the pericardium fixing member is rotated around its axis to rotate the spiral needle-shaped portion, the position of the perforating member made of the linear needle-shaped member, arranged in its center location, relative to the pericardium, does not vary, so that the through-hole may be effortlessly perforated in the pericardium.

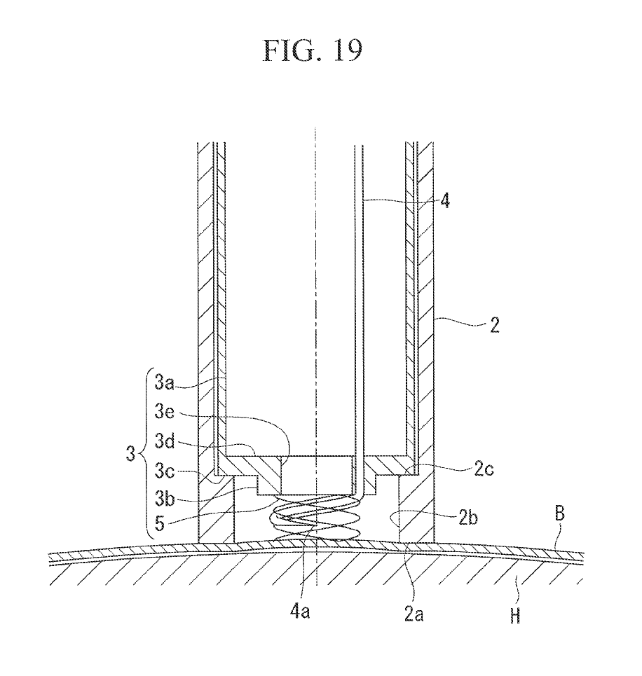

Further, in the present embodiment, the linear cylindrical shape member is illustrated as the perforating member 4, the linear cylindrical shape member being arranged along a direction of the axis D in the center of the pericardium fixing member 3. However, instead of this, as shown in FIG. 19, a cylindrical shape member may be formed into a spiral which is concentric with the spiral needle-shaped portions 5 and wound in the same direction with the same lead, and fixed to the pericardium fixing member 3. Even in this case, the tip 4a of the perforating member 4 is positioned somewhat rearward of the pointed end 5a of the needle-shaped portion 5 in a direction of the axis D.

Thus, the rotation of the pericardium fixing member 3 causes the needle-shaped portions 5 to be stuck in the pericardium B. In the middle of the pericardium B being moved away from the heart H, the tip 4a of the perforating member 4 sticks in the pericardium B. In addition the pericardium fixing member 3 is rotated, thereby proceeding with perforation to the pericardium B by the perforating member 4 to form a through-hole.

Also by doing so, the treatment instrument 1 has an advantage of capable of forming a through-hole for insertion of a guide wire E into the pericardium B without damage to the heart H, and easily inserting the guide wire E into the cardiac sac F.

In other words, when the pericardium fixing member is rotated around its axis so that the spiral needle-shaped portions are stuck in the pericardium, and deeply perforated without changing the location of the perforation, the perforating member made of the spiral needle-shaped member arranged coaxially with the needle-shaped portions also may be stuck in the pericardium to effortlessly form a through-hole.

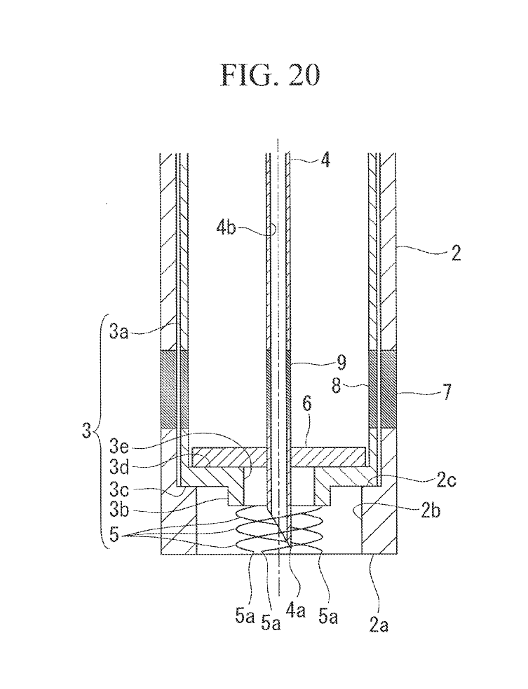

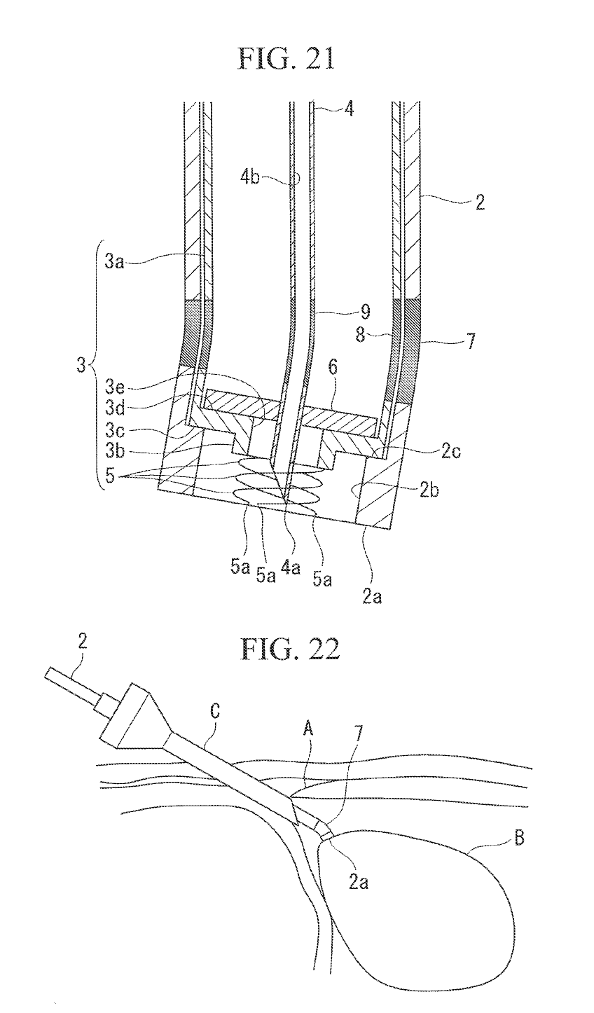

Also, in the present embodiment, as shown in FIGS. 20 and 21, bendable portions 7, 8, 9, which may be bent, may be provided in a part of the outer sheath 2, the body 3a of the pericardium fixing member 3, and the perforating member 4. The bendable portions 7, 8, 9 are constituted of a bendable elastic member such as rubber tubes, bellows, or hoses. In particular, the bendable portion 8 provided in the body 3a of the pericardium fixing member 3 is constituted of a member that may transmit rotational force.

By doing so, in the case where the surface of the pericardium B is inclined in a direction perpendicular to an insertion direction of the outer sheath 2 through the trocar C, as shown in FIG. 22, the bendable portions 7, 8, 9 may be bent to allow the tip surface 2a of the outer sheath 2 to be brought into intimate contact with the inclined surface the of the pericardium B so as to follow it. Therefore, the treatment instrument 1 has an advantage of capable of more surely perforating a through-hole, for insertion of a guide wire E, into a pericardium B that is not perpendicular to an insertion direction of an outer sheath 2.

In other words, even when the insertion direction of the outer sheath does not agree with a normal direction of the outer surface of the pericardium, a bendable portion is bent to direct the tip portions of the outer sheath, the pericardium fixing member, and the perforating member simultaneously in the same direction to allow the tip surface of the outer sheath to be brought into intimate contact with the outer surface of the pericardium. This may more surely perform fixing to a pericardium by the pericardium fixing member and perforation working of a through-hole by the perforating member.

Further, in the present embodiment, although the needle-shaped portions 5 and the spiral perforating member 4 are arranged on the same circumference of a circle, instead of this, the location in radial direction may be different as long as they are arranged coaxially and with the same lead.

As examples shown in FIGS. 20 and 21, although the structure in FIG. 2 with the bendable portions 7, 8, 9 in which the perforating member 4 is movable relative to the pericardium fixing member 3 in a direction of the axis D had been described, instead of this, a structure in FIG. 18 in which the perforating member 4 is fixed to the pericardium fixing member 3 may adopt a bendable portion.

In this way, even when the insertion direction of the outer sheath does not agree with the normal direction of the outer surface of the pericardium, the bendable portion is bent, and the outer sheath and the tip portion of the pericardium fixing member is directed simultaneously in the same direction, to allow the tip surface of the outer sheath to be brought into intimate contact with the outer surface of the pericardium. The perforating member is fixed to the pericardium fixing member, and therefore by bending the pericardium fixing member, the perforating member also may change the direction of the tip simultaneously. This may more surely perform fixing to the pericardium by the pericardium fixing member and perforation working of the through-hole by the perforating member.

Moreover, the present invention provides a guide wire inserting method as described below.

### (Item 1)

A guide wire inserting method, including:

causing a cylindrical sheath to access a pericardium from outside the pericardium;

pressing a tip of the sheath against an outer surface of the pericardium perpendicularly;

inserting fixing unit having a needle-shaped protrusion at a tip into the sheath;

restricting an amount of projection of the needle-shaped protrusion at the tip of the fixing unit relative to the sheath;

rotating the fixing unit around the axis of the sheath relative to the sheath, so as to cause the needle-shaped protrusion to enter the pericardium to raise the pericardium;

moving a needle having a needle hole relative to the fixing unit in a direction of the axis;

restricting an amount of projection of a tip of the needle relative to the fixing unit;

perforating the pericardium by the needle; and

inserting a guide wire through the needle hole of the needle.

### (Item 2)

A guide wire inserting method, including:

causing a cylindrical sheath to access a pericardium from outside the pericardium;

pressing a tip of the sheath against an outer surface of the pericardium perpendicularly;

inserting fixing unit having needle-shaped protrusion and a needle having a needle hole fixed at a tip into the sheath;

restricting an amount of projection of the needle-shaped protrusion and the needle at the tip of the fixing unit relative to the sheath;

rotating the fixing unit around the axis of the sheath relative to the sheath, so as to cause the needle-shaped protrusion to enter the pericardium to raise the pericardium, thereby perforating the pericardium by the needle; and

inserting a guide wire through the needle hole of the needle.

### (Item 3)

The guide wire inserting method according to item 1, further including measuring the thickness of the pericardium before inserting the fixing unit into the sheath.

### (Item 4)

The guide wire inserting method according to item 3, wherein measuring the thickness of the pericardium is measuring the thickness of the pericardium using ultrasonic measurement.

### (Item 5)

The guide wire inserting method according to item 3, wherein measuring the thickness of the pericardium is measuring the thickness of the pericardium using an optical coherence tomography method.

### (Item 6)

The guide wire inserting method according to item 2, further including measuring the thickness of the pericardium before inserting the fixing unit into the sheath.

### (Item 7)

The guide wire inserting method according to item 6, wherein measuring the thickness of the pericardium is measuring the thickness of the pericardium using ultrasonic measurement.

### (Item 8)

The guide wire inserting method according to item 6, wherein measuring the thickness of the pericardium is measuring the thickness of the pericardium using an optical coherence tomography method.

### REFERENCE SIGNS LIST

B pericardium
D axis
E guide wire
H heart
1 treatment instrument
2 outer sheath (guide member)
2a tip surface
2c step portion (pointed end position restricting portion)
3 pericardium fixing member
3a body (cylinder-shaped body)
3c abutment surface (pointed end position restricting portion)
3d step portion (perforating member position restricting portion)
4 perforating member
5 needle-shaped portion
5a pointed end
6 jaw member (perforating member position restricting portion, other abutment surface)
7, 8, 9 bendable portion

1. A treatment instrument, comprising:
a guide member with a tip surface to be pressed against an outer surface of a pericardium;
a pericardium fixing member including at least one needle-shaped portion having a sharp pointed end extending diagonally forward along a circumferential direction at a tip of a cylinder-shaped body rotatable around the axis of the cylinder-shaped body, wherein the cylinder-shaped body is rotated around the axis so as to stick the pointed end of the needle-shaped portion into the pericardium to fix the cylinder-shaped body to the pericardium;
a perforating member for forming a through-hole for a guide wire in the pericardium to which the pericardium fixing member is fixed; and
a pointed end position restricting portion for restricting the position of the pointed end in a direction of the axis relative to the tip surface of the guide member at a location where the pointed end of the pericardium fixing member does not reach the outer surface of a heart while the tip surface of the guide member is pressed against the outer surface of the pericardium.

2. The treatment instrument according to claim 1, further comprising a perforating member position restricting portion for restricting the position of the perforating member in the direction of the axis relative to the tip surface of the guide member at a location where the perforating member does not reach the outer surface of the heart while the tip surface of the guide member is pressed against the outer surface of the pericardium.

3. The treatment instrument according to claim 1, wherein the guide member is formed into a cylindrical shape into which the pericardium fixing member is inserted.

4. The treatment instrument according to claim 1, the needle-shaped portion is formed into a spiral shape.

5. The treatment instrument according to claim 1, wherein
the pericardium fixing member is provided so as to be movable relatively in the direction of the axis with respect to the guide member, and
the pointed end position restricting portion comprises an abutment surface provided on the pericardium fixing member to butt the guide member.

6. The treatment instrument according to claim 2, wherein
the pericardium fixing member is provided so as to be movable relatively in the direction of the axis with respect to the guide member;
the perforating member is provided so as to be movable relatively in the direction of the axis with respect to the guide member or the pericardium fixing member;
the pointed end position restricting portion comprises an abutment surface provided on the pericardium fixing member to butt the guide member; and
the perforating member position restricting portion comprises another abutment surface provided on the perforating member to butt the guide member or the pericardium fixing member.

7. The treatment instrument according to claim 4, wherein the perforating member is fixed to the pericardium fixing member, with a sharp tip of the perforating member arranged posterior to the pointed end of the needle-shaped portion.

8. The treatment instrument according to claim 7, wherein the perforating member is a linear needle-shaped member arranged at the center of the spiral needle-shaped portion along the axis.

9. The treatment instrument according to claim 7, wherein the perforating member is a spiral needle-shaped member arranged coaxially with the needle-shaped portion having the spiral shape.

10. The treatment instrument according to claim 1, wherein the guide member, the pericardium fixing member, and the perforating member are each provided with a bendable portion allowing tip portions of the members to be bent simultaneously in the same direction.

11. The treatment instrument according to claim 7, wherein the guide member and the pericardium fixing member are each provided with a bendable portion allowing tip portions of the members to be bent simultaneously in the same direction.

12. A guide wire inserting method, comprising:
causing a cylindrical sheath to access a pericardium from outside the pericardium;
pressing a tip of the sheath against an outer surface of the pericardium perpendicularly;
inserting fixing unit having a needle-shaped protrusion at a tip into the sheath;
restricting an amount of projection of the needle-shaped protrusion at the tip of the fixing unit relative to the sheath;
rotating the fixing unit around the axis of the sheath relative to the sheath, so as to cause the needle-shaped protrusion to enter the pericardium to raise the pericardium;
moving a needle having a needle hole relative to the fixing unit in a direction of the axis;
restricting an amount of projection of a tip of the needle relative to the fixing unit;
perforating the pericardium by the needle; and
inserting a guide wire through the needle hole of the needle.

13. A guide wire inserting method, comprising:
causing a cylindrical sheath to access a pericardium from outside the pericardium;
pressing a tip of the sheath against an outer surface of the pericardium perpendicularly;
inserting fixing unit having a needle-shaped protrusion and a needle having a needle hole fixed at a tip into the sheath;
restricting an amount of projection of the needle-shaped protrusion and the needle at the tip of the fixing unit relative to the sheath;
rotating the fixing unit around the axis of the sheath relative to the sheath, so as to cause the needle-shaped protrusion to enter the pericardium to raise the pericardium, thereby perforating the pericardium by the needle; and
inserting a guide wire through the needle hole of the needle.

14. The guide wire inserting method according to claim 12, further comprising measuring the thickness of the pericardium before inserting the fixing unit into the sheath.

15. The guide wire inserting method according to claim 14, wherein measuring the thickness of the pericardium is measuring the thickness of the pericardium using ultrasonic measurement.

16. The guide wire inserting method according to claim 14, wherein measuring the thickness of the pericardium is measuring the thickness of the pericardium using an optical coherence tomography method.

17. The guide wire inserting method according to claim 13, further comprising measuring the thickness of the pericardium before inserting the fixing unit into the sheath.

18. The guide wire inserting method according to claim 17, wherein measuring the thickness of the pericardium is measuring the thickness of the pericardium using ultrasonic measurement.

19. The guide wire inserting method according to claim 17, wherein measuring the thickness of the pericardium is measuring the thickness of the pericardium using an optical coherence tomography method.
